(19) United States
(12) Patent Application Publication
(10) Pub. No.: US 2023/0263462 A1
(43) Pub. Date: Aug. 24, 2023

(54) IMPEDANCE BASED WOUND HEALING MONITOR

(71) Applicant: 3M INNOVATIVE PROPERTIES COMPANY, St. Paul, MN (US)

(72) Inventors: Stephen M. Kennedy, St. Paul, MN (US); Jacob E. Odom, Savage, MN (US); Jeffrey P. Kalish, St. Paul, MN (US); Sean M. Sweetnam, Minneapolis, MN (US); Michael D. Benson, Mendota Heights, MN (US); Jana Ninkovic, St. Paul, MN (US); Devon M. Hunerdosse, St. Paul, MN (US); Jason W. Bjork, Cottage Grove, MN (US)

(21) Appl. No.: 18/009,056

(22) PCT Filed: May 25, 2021
(86) PCT No.: PCT/IB2021/054553

§ 371 Date: Dec. 8, 2022

Related U.S. Application Data
(60) Provisional application No. 62/705,072, filed on Jun. 10, 2020.

(51) Int. Cl. A61B 5/00; A61B 5/0531

(52) U.S. Cl. CPC A61B 5/445; A61B 5/0531; A61B 5/6801

(57) ABSTRACT

A method and system for tissue impedance measurement are disclosed. In examples, the system comprises electrical contacts configured to be coupled to a first tissue and a first device configured to apply a first electrical signal to the first tissue via the electrical contacts. The system further comprises a second device configured to determine a first impedance phase angle of epithelial tissue of the first tissue site based on the first applied electrical signal, determine a baseline impedance phase angle of epithelial tissue corresponding to a second tissue, determine information indicative of epithelial tissue characteristics based on a ratio of the first impedance phase angle and the baseline impedance phase angle, and output information indicative of the epithelial tissue characteristics.

### TECHNICAL FIELD

The disclosure relates to wound monitoring.

### BACKGROUND

Smart wound dressings and other wearable sensors are used to monitor healing. The ability to monitor healing may lead to improved healthcare and improved patient outcomes. Monitoring healing may be used to determine whether a current treatment program is effective, or whether changes to a current treatment program should be made.

### SUMMARY

In general, the disclosure describes example techniques for determining and/or tracking physiological measures of wound healing using electrical signals, more specifically, the phase angle of impedance at one or more locations at a wound site.

One or more pairs of electrodes may apply an electrical signal, or signals, to a tissue site, or a location within a tissue site, and processing circuitry may determine an impedance phase angle of the wounded tissue at the location based on the applied electrical signal or signals. The tissue site may correspond to a wound, e.g., tissue having damage. In some examples, the processing circuitry determines a baseline impedance phase angle (e.g., impedance phase angle at unwounded location, or pre-stored impedance phase angle information such as expected impedance phase angle based on average of patients). Information indicative of epithelial tissue characteristics may be determined based on the impedance phase angle of the wounded tissue or both the baseline impedance phase angle and the impedance phase angle of the wounded tissue.

The techniques disclosed may provide simplified, low-cost techniques capable of providing real-time, clinically actionable output related to healing, or lack thereof. For instance, utilizing impedance phase angles may provide a more accurate measure of the patient healing as compared to other techniques. Continuous monitoring and capture of real-time wound healing data may lead to improved healthcare and improved patient outcomes.

In some examples, the disclosure describes a system for tissue impedance measurement, the system comprising: electrical contacts configured to be coupled to a first tissue; a first device configured to apply a first electrical signal to the first tissue via the electrical contacts; and a second device configured to: determine a first impedance phase angle of epithelial tissue of the first tissue site based on the first applied electrical signal; determine a baseline impedance phase angle of epithelial tissue corresponding to a second tissue; determine information indicative of epithelial tissue characteristics based on a ratio of the first impedance phase angle and the baseline impedance phase angle; and output information indicative of the epithelial tissue characteristics.

In some examples, the disclosure describes a method, comprising: applying a first electrical signal through a first tissue via electrical contacts; determining a first impedance phase angle of the first tissue based on the first applied electrical signal; determining a baseline impedance phase angle corresponding to a second tissue; determining information indicative of epithelial tissue characteristics based on a ratio of the first impedance phase angle and the baseline impedance phase angle; and outputting information indicative of the tissue characteristics.

In some examples, the disclosure describes a dressing, comprising: electrical contacts configured to be coupled to wounded tissue and to unwounded tissue proximate to wounded tissue; a first device configured to apply a first electrical signal through the wounded tissue via the electrical contacts, the first device further configured to apply a second electrical signal through the unwounded tissue; and a second device configured to: determine a first impedance phase angle of the tissue based on the first applied electrical signal; determine a second impedance phase angle off the tissue based on the second applied electrical signal; determine information indicative of epithelial tissue characteristics based on a ratio of the first impedance phase angle and the second impedance phase angle; and output information indicative of the tissue characteristics.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is an illustration depicting an example wound monitoring system, in accordance with the techniques described in this disclosure.

FIG. 2A is a conceptual diagram illustrating an aerial view an example tissue site, in accordance with one or more techniques of this disclosure.

FIG. 2B is a conceptual diagram illustrating a side-view of the example tissue site of FIG. 2A, in accordance with one or more techniques of this disclosure.

FIG. 3A is a conceptual diagram illustrating an aerial view an example tissue site, in accordance with one or more techniques of this disclosure.

FIG. 3B is a conceptual diagram illustrating a side-view of the example tissue site of FIG. 3A, in accordance with one or more techniques of this disclosure.

FIG. 4 is an illustration depicting an example wound monitoring system, in accordance with the techniques described in this disclosure.

FIG. 5 is a plot of example impedance phase angles as a function of signal frequency for first and second tissue sites, in accordance with the techniques described in this disclosure.

FIG. 6 is a plot of example impedance magnitudes as a function of signal frequency for first and second tissue sites, in accordance with the techniques described in this disclosure.

FIG. 7 is a plot of an example correlation between normalized impedance phase angle at a single frequency and a percentage of new epithelial monolayer, in accordance with the techniques described in this disclosure.

FIG. 8 is a plot of example normalized impedance phase angles of wounded tissues versus time as eight wounds heal on two animals, in accordance with the techniques described in this disclosure.

FIG. 9A is a plot of example predicted epithelial cross-sectional areas as a function of time based on the normalized impedance phase angles versus time of FIG. 8, in accordance with the techniques described in this disclosure.

FIG. 9B is a plot of example measured epithelial cross-sectional areas at day seven of FIG. 8 based on histology, in accordance with the techniques described in this disclosure.

FIG. 9C is a plot of an example correlation between normalized impedance phase angle at a single frequency and epithelial cross-sectional area, in accordance with the techniques described in this disclosure.

FIG. 10A is a plot of example predicted average epithelial depths as a function of time based on the normalized impedance phase angles versus time of FIG. 8, in accordance with the techniques described in this disclosure.

FIG. 10B is a plot of example measured average epithelial depths at day seven of FIG. 8 based on histology, in accordance with the techniques described in this disclosure.

FIG. 10C is a plot of an example correlation between normalized impedance phase angle at a single frequency and average epithelial depth, in accordance with the techniques described in this disclosure.

FIG. 11A is a plot of example predicted corneal site de-nucleation percentages as a function of time based on the normalized impedance phase angles versus time of FIG. 8, in accordance with the techniques described in this disclosure.

FIG. 11B is a plot of example measured corneal site de-nucleation percentages at day seven of FIG. 8 based on histology, in accordance with the techniques described in this disclosure.

FIG. 11C is a plot of an example correlation between normalized impedance phase angle at a single frequency and a percentage of corneal site de-nucleation, in accordance with the techniques described in this disclosure.

FIG. 12 is a flowchart of an example method of monitoring wound healing, in accordance with one or more techniques of this disclosure.

### DETAILED DESCRIPTION

In examples, the disclosure describes a method and system for tracking quantitative metrics of wound healing by measuring the impedance phase angle of wound beds versus time. In general, a wound may include tissue having damage, bruised tissue, burned tissue, scraped tissue, tissue having a rash, and the like. However, the electrical characteristics of tissues have not been well-characterized as a wound is generated and as it undergoes healing. This has limited the development of robust monitoring strategies.

Tissue impedance phase angle, in particular, may undergo characteristic changes from injury through healing, and this change versus time may be used to quantitatively track wound healing. For example, unwounded tissues may exhibit capacitive charging over a certain range of frequencies. That is, unwounded tissues comprise epithelial layers with high cell densities and tight cell-cell junctions which serve as a barrier upon which charge will accumulate. As such, the top layers of tissues act as a parallel plate capacitor. This capacitance will manifest as a complex impedance with a phase angle θ between -90° and 0° over a range of frequencies associated with the charging timescales of the epithelial layer. However, when wounded, these tissue layers can be damaged or removed, reducing the tissue’s inherent electrical capacitance. This loss of capacitance will manifest as a shorting of the complex impedance’s phase angle towards 0° over this range in frequencies. As the wound begins to heal, tightly joined epithelial layers are gradually restored, first by ingrowth of an epithelial monolayer, then a thickening of this monolayer through epithelial proliferation, and then with a de-nucleation of cells contained in the epithelial layer’s topmost cornea. The restoration of the tightly joined epithelial layers shifts the phase angle over the range of frequencies back to between -90° and 0°. Thus, monitoring impedance phase angle versus time may indicate the amount of healing experienced in the wound bed and provide a technique for tracking one parameter, e.g., impedance phase angle, indicative of physiologically relevant and quantitative metrics of healing. Impedance phase angle may be measured by injecting sinusoidal signals into the tissue and measuring the phase shift between the signals’ voltages and currents.

In general, tracking impedance phase angle may require relatively few components and can be measured with relatively low-cost components. Accordingly, this disclosure provides a way to determine how much a wound has healed using relatively few low-cost components that may be integrated into a system.

The ability to monitor wound healing without removing a dressing material (which can significantly disrupt the wound bed) may be a powerful tool, particularly if the data are indicative of quantitative, physiological metrics of healing. For example, simply knowing if (and to what degree) a wound is healing or not will inform clinicians or the patient/customer if intervention is required or not (e.g., changing the dressing, cleaning the wound, administering therapeutics to the wound). Monitoring of wound healing through impedance phase angle may reduce the need for frequent dressing changes. For example, with each dressing removal the tissue gets traumatized and healing gets delayed. By monitoring wounds, the frequency of dressing changes may be reduced, thereby reducing tissue trauma and pain and improving healing over the duration of treatment. The need for non-complex and continuous wound monitoring is more salient in chronic wound management. Chronic wounds pose a major threat to public health and healthcare economies. Chronic wounds are also most prevalent in rapidly expanding populations of elderly, obese, and diabetic patients.

In examples, measurement of impedance phase angle correlates strongly to several physiological metrics of healing associated with the continuity, size, and degree of maturation of an epithelial layer regenerated over the wound. Measurements of the impedance phase angle may detect delays in healing and may detect differences in healing rate/quality between tissues exhibiting different degrees of healing, e.g., between different humans and/or different animals. Measurement of impedance phase angle of a wound site and/or location may be robust across multiple wound sites and across different animals, in contrast with impedance magnitude. The methods disclosed may be implemented with non-complex, low-cost electronics which could be integrated directly into “smart” wound dressings capable of providing real-time, clinically actionable readouts of healing (or a lack of healing, e.g., when detecting chronic wounds).

In examples, one or more pairs of electrodes may apply an electrical signal, or signals, to a wound site, or a location within a wound site, and processing circuitry may determine an impedance phase angle of the wounded tissue at the location based on the applied electrical signal or signals. Additionally, one or more pairs of electrodes may apply an electrical signal, or signals, to a location proximate to the wound site, e.g., an unwounded location, and determine a baseline impedance phase angle at the unwounded location based on the applied electrical signal or signals. Alternatively, in some examples, the processing circuitry determines a baseline impedance phase angle (e.g., impedance phase angle at unwounded location, or pre-stored impedance phase angle information such as expected impedance phase angle based on average of patients, patients of particular demographics, and/or the anatomical location of the wound). Information indicative of epithelial tissue characteristics may be determined based on the impedance phase angle of the wounded tissue or both the baseline impedance phase angle and the impedance phase angle of the wounded tissue.

In some examples, information indicative of epithelial tissue characteristics may be determined based on the impedance phase angle, or angles, and without using the impedance magnitude. In some examples, the electrodes may apply a single-frequency electrical signal and determine impedance phase angles at the single frequency based on the applied electrical signal. Information indicative of epithelial tissue characteristics may be determined based on single-frequency impedance phase angles. In other examples, electrical signals comprising a band of frequencies, or comprising a broad spectrum or frequency sweep, may be applied, impedance phase angles may be determined based on the multi-frequency signals, and information indicative of epithelial tissue characteristics may be determined based on multi-frequency impedance phase angles.

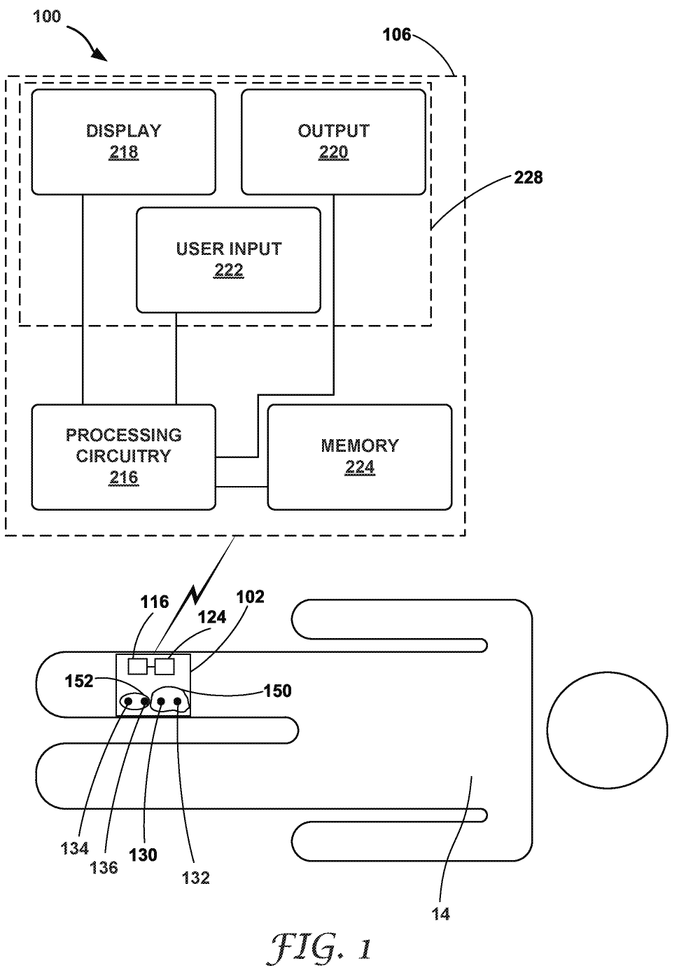

FIG. 1 is an illustration depicting an example wound monitoring system 100, in accordance with the techniques described in this disclosure. In the example shown, wound monitoring system 100 includes dressing 102 and computing device 106. Dressing 102 may be communicatively coupled, for example by a wired or a wireless connection, to computing device 106. In the illustrated example, computing device 106 may include processing circuitry 216 coupled to display 218, output 220, and user input 222 of a user interface 228.

In some examples, dressing 102 may be configured to apply electrical signals to tissue site 150 of patient 14, apply electrical signals proximate to tissue site 150, e.g., at second tissue site 152, and to detect electrical signals applied to tissue site 150 and proximate to tissue site 150. In some examples, first tissue site 150 may correspond to wounded tissue, e.g., tissue having damage to epithelial layers. In some examples, second tissue site 152 may correspond to tissue without damage, e.g., healthy tissue. In other examples, first tissue site 150 may correspond to tissue having a bruise, tissue having a rash, tissue having an infection, and the like. The electrical signals, or information corresponding to the electrical signals, may be transferred to computing device 106 for processing, for example, by a wired or wireless connection between dressing 102 and computing device 106. In some examples, dressing 102 may include processing circuitry 116 and memory 124 and may process the electrical signals without transferring the electrical signals to computing device 106.

Dressing 102 may be any type of structure that includes any of electrodes 130-136, and in some examples may comprise just electrodes 130 and 132. In some examples, dressing 102 may be a bandage including a flexible backing material, an adhesive for bonding to the skin of patient 14, and electrodes 130-136. In some examples, dressing 102 may be a foam dressing including electrodes 130-136. In other examples, dressing 102 may be a diagnostic patch, for example, a material including any of electrodes 130-136. In some examples, additional materials may be applied to patient 14 for wound monitoring, for example, sterile saline-laden gauze, a gel, or the like, placed between dressing 102 and tissue site 150. In some examples, additional materials may include treatments such as medications and/or may be at least partially electrically conductive and may enhance electrical conductivity between electrodes 130-136 and tissue site 150 and second tissue site 152.

Electrodes 130-136 may be any type of conductors capable of conducting electrical signals. For example, electrodes 130-136, alternatively referred to as electrical contacts 130-136, may be configured to apply an electrical signal to a material with which they are in contact, e.g., the skin of patient 14. Electrodes 130-136 may in addition be configured to detect, sense, capture, etc., electrical signals from a material with which they are in contact. In some examples, electrodes 130-136 may apply and detect electrical signals to/from a material in a non-contact manner, e.g., via inductance, capacitive coupling, and/or electromagnetic radiation. In some examples, electrodes 130-136 are configured to apply and detect signals in a wide frequency range. For examples, electrodes 130-136 may be configured to apply and detect electrical signals having frequencies between 1 kHz to 2 kHz, between 2 kHz to 4 kHz, between 4 kHz to 55 kHz, between 55 kHz to 120 kHz, or having frequencies in ranges that may be greater or less than these example ranges. In some examples, electrodes 130 may be configured to apply and detect electrical signals having any frequency.

In some examples, electrodes 130 and 132 are configured to be in contact with tissue site 150 and to conduct electrical signals to and from wounded tissue, e.g., as an electrode pair. In some examples, electrodes 134 and 136 are configured to be in contact with tissue adjacent to tissue site 150, e.g., second tissue site 152, and to conduct electrical signals to and from the adjacent tissue, e.g., as an electrode pair. In some examples, electrodes 130 and 132 are different from electrodes 134 and 136, e.g., so as to be able to conduct electrical signals to and from both unwounded and wounded tissue at approximately the same time. In some examples, electrodes 130 and 132 may be the same as electrodes 134 and 136, e.g., so as to be able to conduct electrical signals to and from both unwounded and wounded tissue using the same electrodes via moving the electrodes.

Processing circuitry 216 of computing device 106, as well as processing circuitry 116 and other processing modules or circuitry described herein, may be any suitable software, firmware, hardware, or combination thereof. Processing circuitry 216 may include any one or more microprocessors, controllers, digital signal processors (DSPs), application specific integrated circuits (ASICs), field-programmable gate arrays (FPGAs), or discrete logic circuitry. The functions attributed to processors described herein, including processing circuitry 216, may be provided by processing circuitry of a hardware device, e.g., as supported by software and/or firmware.

In some examples, processing circuitry 216, as well as processing circuitry 116, is configured to determine physiological information and/or information indicative of epithelial tissue characteristics associated with patient 14. For example, processing circuitry 216 may determine an impedance, and/or impedances, of tissue site 150, based on applied electrical signals. In some examples, processing circuitry 216 may determine an impedance, and/or impedances, of a location proximate to tissue site 150 based on applied electrical signals. In some examples, processing circuitry 216 may determine one or more impedance phase angles of tissue site 150, a location within tissue site 150, and/or a location proximate to tissue site 150. In some examples, processing circuitry 216 may determine one or more impedance magnitudes of tissue site 150, a location within tissue site 150, and/or a location proximate to tissue site 150. In some examples, processing circuitry 216 may determine information indicative tissue characteristics based on the impedance, impedance magnitude, and/or impedance phase angle. In some examples, processing circuitry 216 may determine information indicative tissue characteristics based on the impedance phase angle without determining or using impedance magnitude. For example, processing circuitry may determine information indicative of epithelial tissue characteristics such as wound healing, e.g., an amount of new epithelial monolayer regenerated in tissue, an amount of size or thickness of regenerated epithelium, a relative maturation of stratus corneum, and/or an amount of corneal site de-nucleation.

Processing circuitry 216 may perform any suitable signal processing of electrical signals to filter the electrical signals, such as any suitable band-pass filtering, adaptive filtering, closed-loop filtering, any other suitable filtering or processing as described herein, and/or any combination thereof. Processing circuitry 216 may also receive input signals from additional sources (not shown). For example, processing circuitry 216 may receive an input signal containing information about treatments provided to the patient. Additional input signals may be used by processing circuitry 216 in any of the calculations or operations it performs in accordance with wound monitoring system 100. In some examples, processing circuitry 216 may be adapted to execute software, which may include an operating system and one or more applications, as part of performing the functions described herein. In some examples, processing circuitry 216 may include one or more processing circuitry for performing each or any combination of the functions described herein.

In some examples, processing circuitry 216 may be coupled to memory 224, and processing circuitry 116 may be coupled to memory 124. Memory 224, as well as memory 124, may include any volatile or non-volatile media, such as a random-access memory (RAM), read only memory (ROM), non-volatile RAM (NVRAM), electrically erasable programmable ROM (EEPROM), flash memory, and the like. Memory 224 may be a storage device or other non-transitory medium. Memory 224 may be used by processing circuitry 216 to, for example, store fiducial information or initialization information corresponding to physiological monitoring, such as wound monitoring. In some examples, processing circuitry 216 may store physiological measurements or previously received data from electrical signals in memory 224 for later retrieval. In some examples, processing circuitry 216 may store determined values, such as information indicative of epithelial tissue characteristics, or any other calculated values, in memory 224 for later retrieval.

Processing circuitry 216 may be coupled to user interface 228 including display 218, user input 222, and output 220. In some examples, display 218 may include one or more display devices (e.g., monitor, PDA, mobile phone, tablet computer, any other suitable display device, or any combination thereof). For example, display 218 may be configured to display physiological information and information indicative of epithelial tissue characteristics determined by wound monitoring system 100. In some examples, user input 222 is configured to receive input from a user, e.g., information about patient 14, such as age, weight, height, diagnosis, medications, treatments, and so forth. In some examples, display 218 may exhibit a list of values which may generally apply to patient 14, such as, for example, age ranges or medication families, which the user may select using user input 222.

User input 222 may include components for interaction with a user, such as a keypad and a display, which may be the same as display 218. In some examples, the display may be a cathode ray tube (CRT) display, a liquid crystal display (LCD) or light emitting diode (LED) display and the keypad may take the form of an alphanumeric keypad or a reduced set of keys associated with particular functions. User input 222, additionally or alternatively, include a peripheral pointing device, e.g., a mouse, via which a user may interact with the user interface. In some examples, the displays may include a touch screen display, and a user may interact with user input 222 via the touch screens of the displays. In some examples, the user may also interact with user input 222 remotely via a networked computing device.

In some examples, wound monitoring system 100 may determine information indicative of healing. For example, processing circuitry 116 and/or 216 may determine information indicative of epithelial tissue characteristics such as wound healing based on impedance phase angle. In some examples, determination of wound healing may be based on impedance phase angle of wounded tissue normalized, e.g., divided by, a baseline impedance phase angle. In other words, determination of wound healing may be based on a ratio of impedance phase angle of wounded tissue and a baseline impedance phase angle. In some examples, determination of wound healing and/or information indicative of epithelial tissue characteristics may be based on a plurality of ratios of impedance phase angles of wounded tissue and one or more baseline impedance phase angles.

For example, impedance phase angle may vary because of variation in the electrical characteristics from tissue site to tissue site of an individual, e.g., different tissue locations on the same patient and/or animal, variation of tissue characteristics at different times, and variation from individual to individual, e.g., from patient to patient and/or animal to animal. For example, electrical characteristics of tissue may vary based on tissue composition and thickness, tissue water content and/or tissue hydration, ambient relative humidity at the time of measurement, and the like. In some examples, to reduce site to site and/or patient to patient impedance phase angle variation, a baseline impedance phase angle may be determined at or near the time of measurement of an impedance phase angle of first tissue, e.g., wounded tissue, and may be used to normalize the first tissue impedance phase measurement. For example, a tissue baseline impedance phase angle of a second tissue, e.g., unwounded tissue, may be measured concurrently at a second tissue site which may be adjacent to the first tissue, and may be used to normalize the first tissue impedance phase angle measurement.

However, measuring the baseline impedance phase angle concurrently or near concurrently with the impedance phase angle of the wounded tissue is not necessary in all examples. In some examples, a baseline impedance phase angle representative of unwounded tissue corresponding to the wounded tissue site, e.g., tissue site 150, may be measured previous to measurement of an impedance phase angle of wounded tissue at tissue site 150, and in some examples may be electronically stored for future normalization, e.g., after a wound occurs at tissue site 150. In still other examples, a baseline impedance phase angle may be derived from previously measured impedance phase angles of unwounded tissue of a representative population, e.g., an average of measured impedance phase angles of unwounded tissues at particular anatomical sites of a population of patients. In still other examples, a baseline impedance phase angle may be theoretically derived, or derived by any other appropriate techniques for representing a baseline impedance phase angle of unwounded tissue that corresponds to wounded tissue.

Generally, impedance is a complex quantity including what are referred to as “real” and “imaginary” quantities, e.g., Z = R + iX, where Z is the impedance, R is the so-called real component and is the resistance, and X is the so-called imaginary component and is the reactance. The magnitude of the impedance may be calculated as the modulus of Z,

$$\text{e} \text{.q} \text{.,} \left|\text{Z}\right| = \sqrt{Z Z ∗} =$$

$$\sqrt{{R}^{2} + {X}^{2}} ,$$

where Z* is the complex conjugate of Z. The impedance phase angle may be calculated as

$$\text{θ=} {\text{tan}}^{- 1} \left(\frac{X}{R}\right) ,$$

where θ is the impedance phase angle. An electrical component with a finite reactance X, such as unwounded tissue that may act as a capacitor, induces a phase shift θ between a voltage across the component and the current through the component. For example, an impedance phase angle may be a value between 0 and -90°, or equivalently between 0 and -π radians. In other examples, an impedance phase angle may be a value between -180° and 180°, or equivalently between -π and π radians. In still other examples, an impedance phase angle may be a value between -360° and 360°, or equivalently between -2π and 2π radians.

In some examples, processing circuitry may determine a ratio between an impedance phase angle of wounded tissue and a baseline impedance phase angle, both angles being in degrees or both angles being in radians, and the resulting value may be indicative of healing. In some examples, the magnitudes of the wounded tissue impedance and baseline impedance may not be needed to calculate the ratio. In other words, the ratio on which information indicative of epithelial tissue characteristics and/or wound healing may depend on wounded tissue impedance phase angle and baseline impedance phase angle and without wounded tissue impedance magnitude or baseline impedance magnitude.

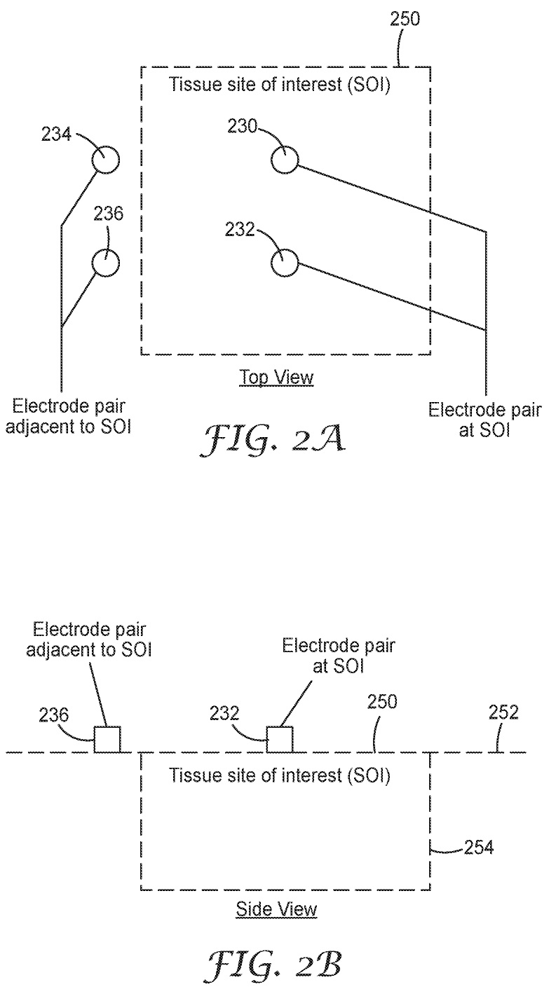

FIG. 2A is a conceptual diagram illustrating an aerial view an example tissue site 250, in accordance with one or more techniques of this disclosure. In the example shown, electrodes 230 and 232 are electrically coupled to tissue within tissue site 250, and electrodes 234 and 236 are electrically coupled to tissue adjacent to tissue site 250, e.g., tissue site 252. In some examples, an alternating electrical signal may be applied to tissue site 250 via electrodes 230 and 232, and an impedance phase angle of the wounded tissue may be determined, for example, via a circuit including electrodes 230 and 232. An alternating electrical signal may also be applied to tissue adjacent to tissue site 250 via electrodes 234 and 236, and a baseline impedance phase angle, e.g., an unwounded tissue impedance phase angle, may be determined, for example, via a circuit including electrodes 234 and 236.

FIG. 2B is a conceptual diagram illustrating a side-view of the example tissue site 250 of FIG. 2A, in accordance with one or more techniques of this disclosure. In the example shown, the electrodes 230-236 are electrically coupled to the outside of tissue surface 252. Tissue site 250 includes a tissue volume 254, e.g., wound bed, extending beneath tissue surface 254. In some examples, any or all of electrodes 236 may be attached to tissue via a dressing, and an at least partially electrically conductive material, gel, medication, and the like may be applied to any or all of electrodes 230-236, e.g., to facilitate and/or improve electrical coupling between electrodes 230-236 and the tissue.

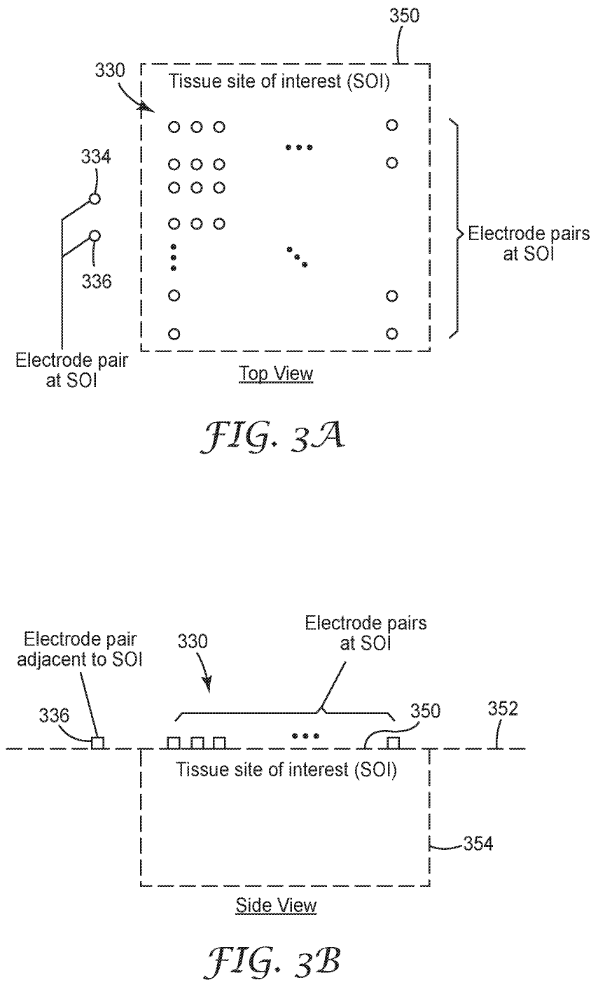

FIG. 3A is a conceptual diagram illustrating an aerial view an example tissue site 350, in accordance with one or more techniques of this disclosure. In the example shown, a plurality of electrodes 330 are electrically coupled to tissue within tissue site 350, and electrodes 334 and 336 are electrically coupled to tissue adjacent to tissue site 350, e.g., tissue site 352. In some examples, an alternating electrical signal may be applied to tissue site 350 via electrodes 330, and a plurality of impedance phase angles of wounded tissue corresponding to a plurality of locations within tissue site 350 may be determined, for example, via one or more circuits including electrodes 330. An alternating electrical signal may also be applied to tissue adjacent to tissue site 350 via electrodes 334 and 336, and a baseline impedance phase angle, e.g., an unwounded tissue impedance phase angle, may be determined, for example, via a circuit including electrodes 334 and 336.

FIG. 3B is a conceptual diagram illustrating a side-view of the example tissue site of FIG. 3A, in accordance with one or more techniques of this disclosure. In the example shown, electrodes 330, 334, and 336 are electrically coupled to the outside of tissue surface 352. Tissue site 350 includes a tissue volume 354, e.g., wound bed, extending beneath tissue surface 354. In some examples, any or all of electrodes 336 may be attached to tissue via a dressing, and an at least partially electrically conductive material, gel, medication, and the like may be applied to any or all of electrodes 330, 334, and 336, e.g., to facilitate and/or improve electrical coupling between electrodes 330, 334, and 336 and the tissue.

In some examples, electrical and/or electronic components and/or circuits may determine an impedance phase angle via measurement of a phase shift between a current and a voltage. In some examples, a reference signal and a measurement signal may be used to determine an impedance phase angle. For example, a phase shift at a frequency f may be ϕ° = (t2-t1)/T * 360° = (t2-t1)*f* 360°, where t1 is the time when a reference signal crosses a certain reference point, ‘t2’ is the time when a measurement signal crosses the same reference point, and T is period of the signal, and 1/T is the frequency of the signal, f. In radians, ϕ = (t2-t1)/T * 2π = (t2-t1)*f*2π.

In some examples, a circuit to measure and/or detect an impedance phase angle including any of electrodes 130-136, or 230-236, or 330-336 may include analog and/or digital circuit components, e.g., resistors, inductors, capacitors, filters, operational amplifiers, analog comparators, analog frequency mixers, digital phase detectors, logic components (AND gates, OR gates, NAND gates, XOR gates and the like), analog-to-digital (A/D) converters, digital-to-analog (D/A converters), microcontrollers, and the like.

For examples, a circuit may determine an impedance phase angle using an analog frequency mixer to determine a phase shift between a current and a voltage. The output of a frequency mixer may be proportional a phase difference between a voltage and a current when two mixer inputs are the same frequency. A frequency mixer may be used in a phase locked loop for frequency synthesis to determine phase shift.

In other examples, a digital phase detector may determine an impedance phase angle. For example, a reference comparator integrated circuit (IC) may receive a reference signal and a measurement signal comparator IC may receive a measurement signal. The reference comparator IC and the measurement signal comparator may set comparator switching levels to the reference signal voltage midpoint. The two IC comparators’ outputs may be coupled to the two inputs of a XOR (Exclusive OR) gate IC. The XOR gate may output a pulse having width substantially equal to (t2-t1), e.g., a value in seconds for the time difference of (t2-t1). The measured time difference (t2-t1) may be used to calculate the phase angle between the two signals.

In some examples, digital phase detector and a microcontroller may determine an impedance phase angle. For example, a microcontroller IC may generate a reference signal and perform phase comparison with a measurement signal. In some examples, an analog comparator including a 6-bit D/A converter may be used to generate the comparator reference signal. For a given measurement cycle of the microcontroller, the D/A converter may generate a sine wave at a reference measurement frequency. Reference timers on the microcontroller may be started and the measurement signal may be passed through the media to be measured, e.g., via electrodes 130-136, 230-236, and/or 330-336. A predetermined number of samples of the reference signal may be compared to the predetermined number of samples of the measurement signal using the microcontroller. The reference and measurement samples may be averaged a predetermined number of times. Using the averaged reference and measurement signals, a time difference, Δt = (t2-t1), may be calculated, and a phase shift may be calculated based on the time difference.

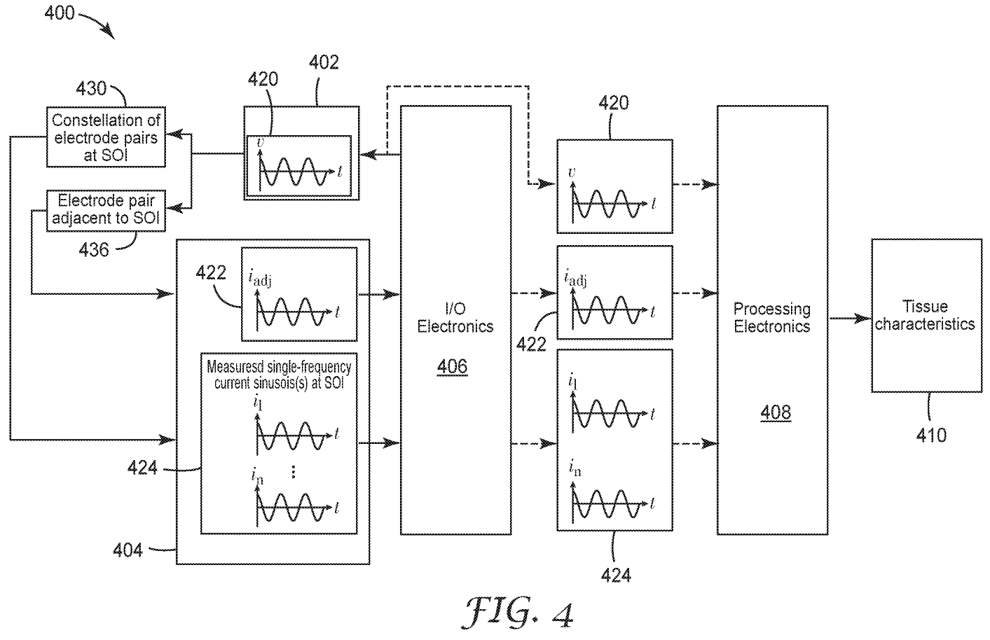

FIG. 4 is a block diagram of an example wound monitoring system 400, in accordance with the techniques described in this disclosure. In the example shown, wound monitoring system 400 includes signal generator 402, tissue site electrodes 430, second tissue site electrodes 436, signal monitor 404, input-output (I/O) electronics 406, processing circuitry 408, and output 410. Wound monitoring system may be an example of wound monitoring system 100 illustrated and described above with respect to FIG. 1.

In the example shown, signal generator 402 is configured to generate an alternating electrical signal, e.g., an electrical waveform. The electrical signal may be sinusoidal, a square wave, a pulse wave, a triangle wave, a sawtooth wave, and the like. Signal generator 402 may be configured to generate an electrical signal including one or more frequencies at any frequency, including between 1 kHz to 2 kHz, between 2 kHz to 4 kHz, between 4 kHz to 55 kHz, between 55 kHz to 120 kHz. In some examples, signal generator 402 may be configured to generate an electrical signal in a frequency range that may be greater than or less than the example ranges above. In some examples, electrical signal generator 402 may be configured to generate an electrical signal at a single frequency, such as approximately 100 kHz (e.g., 100 kHz ± 10 kHz). In the example shown, signal generator 402 is configured to generate voltage signal 420.

In the example shown, signal generator 402 is electrically connected to tissue site electrodes 430 and second tissue site electrodes 436. Tissue site electrodes 430 may include two or more electrodes, and may be substantially similar to electrodes 130, 132, 230, 232, and 330 described above. Second tissue site electrodes 436 may include two or more electrodes, and may be substantially similar to electrodes 134, 136, 234, 236, 334, and 336 described above. Tissue site electrodes 430 and second tissue site electrodes 436 may be configured to be electrically connected to tissue, and in some examples may be attached to tissue via a dressing. Signal generator 402 may be an example of at least a portion of processing circuitry 408 described below. Signal generator 402 may be an example of at least a portion of processing circuitry 116 and/or 216 illustrated and described above, with respect to FIG. 1.

In the example shown, signal monitor 404 is electrically connected to tissue site electrodes 430 and second tissue site electrodes 436. In some examples, signal monitor 404 may be configured to determine and/or measure electrical signals, e.g., voltage signals, current signals, and the like, and signal monitor 404 may be configured to determine and/or measure electrical resistance, electrical impedance, electrical impedance phase angle, and the like. For example, signal monitor 404 may be configured to measure an impedance phase angle of tissue between any two electrodes of tissue site electrodes 430, e.g., via measurement of a voltage signal and corresponding current signal of a circuit including signal generator 402, electrodes 430, and tissue to which electrodes 430 are electrically connected. In some examples, signal monitor 404 is configured to measure a plurality of impedance phase angles corresponding to a plurality of locations of tissue two which electrodes 430 are electrically connected, e.g., a tissue site.

In the example shown, signal monitor 404 is configured to measure current signal 422 via electrodes 436 and one or more current signals 424 via electrodes 430. In some examples, signal monitor 404 may derive an impedance phase angle from an impedance measurement, and in other examples an impedance phase angle may be derived by a different device receiving information corresponding to an impedance measurement by signal monitor 404, e.g., derived by processing circuitry 408 described below. In some examples, signal monitor 404 may measure and/or determine an impedance angle of unwounded tissue, e.g., a baseline impedance phase angle. For example, signal monitor 404 may measure and/or determine an impedance phase angle of tissue between two or more electrodes of second tissue site electrodes 436. Signal monitor 404 may be an example of at least a portion of processing circuitry 408 described below. Signal monitor 404 may be an example of at least a portion of processing circuitry 116 and/or 216 illustrated and described above, with respect to FIG. 1.

In some examples, signal generator 402 may be electrically connected to only tissue site electrodes 436, and signal monitor 404 may determine one or more wounded tissue impedance phase angles. A baseline impedance phase angle, e.g., of unwounded tissue, may be pre-stored, such as an expected impedance phase angle based on an average impedance phase angle of unwounded tissue from a patient population.

In some examples, signal generator 402 and signal monitor 404 may be configured to generate and measure electrical signals at a plurality of frequencies such that a plurality of impedance phase angles corresponding to the plurality of frequencies may be determined and/or measured. In other words, wound monitoring system 400 may determine wounded tissue impedance phase angles and baseline impedance phase angles as a function of frequency, e.g., via a frequency sweep.

In the example shown, I/O electronics 406 is communicatively coupled to signal generator 402, signal monitor 404, and processing circuitry 408. In some examples, I/O electronics may be included in computing device 106 illustrated and described with respect to FIG. 1 above and may be configured as an interface between electrical devices, e.g., signal generator 402, signal monitor 404, and processing circuitry 408. For example, I/O electronics 406 may be configured to communicate signal generation commands and information from processing circuitry 408 to signal generator 402, and receive information corresponding to generated electrical signals from signal generator 402 and relay the received information to processing circuitry 408. I/O electronics 406 may receive information corresponding to measurements, electrical signals and/or impedance phase angles from signal monitor 404 and relay the received information to processing circuitry 408. In the example of FIG. 4, I/O electronics 406 is configured to relay information corresponding to generated voltage signal 420 and measured current signals 422 and 424 to processing circuitry 408.

In some examples, processing circuitry 408 is configured to receive electrical signals, and/or information corresponding to electrical signals, and determine impedance phase angles based on the received information and/or signals. Processing circuitry 408 may also be configured to determine information indicative of epithelial tissue characteristics based on impedance phase angles. In some examples, to improve determination of information indicative of epithelial tissue characteristics, processing circuitry 408 may be configured to determine a ratio of impedance phase angles to baseline impedance phase angles. For example, processing circuitry 408 may determine information indicative of epithelial tissue characteristics based on a ratio of impedance phase angles of wounded tissue, e.g., corresponding to tissue locations to which electrodes 430 are electrically connected, to a baseline impedance phase angle, e.g., a pre-stored baseline impedance phase angle, a measured unwounded tissue impedance phase angle corresponding to tissue locations to which second tissue site electrodes 436 are electrically connected. In some examples, processing circuitry may be configured to determine information indicative of an amount of healing, an amount of new epithelial coverage regenerated in tissue, and amount of size or thickness of regenerated epithelium, a relative maturation of stratus corneum, and the like, based on impedance phase angles.

Processing circuitry 408 may be an example of processing circuitry 216 or 116 illustrated and described above with respect to FIG. 1.

In the example shown, processing circuitry is communicatively coupled to output 410. In some examples, output 410 may be substantially similar to user interface 228 of computing device 106, illustrated and described above with respect to FIG. 1. In some examples, output 410 may be configured to communicate, record, and or display values and/or information indicative of epithelial tissue characteristics, e.g., to a user, a database, network, another computing device, and the like.

In some examples, any and/or all of signal generator 402, signal monitor 404, I/O electronics 406, and processing circuitry may be included in a single device.

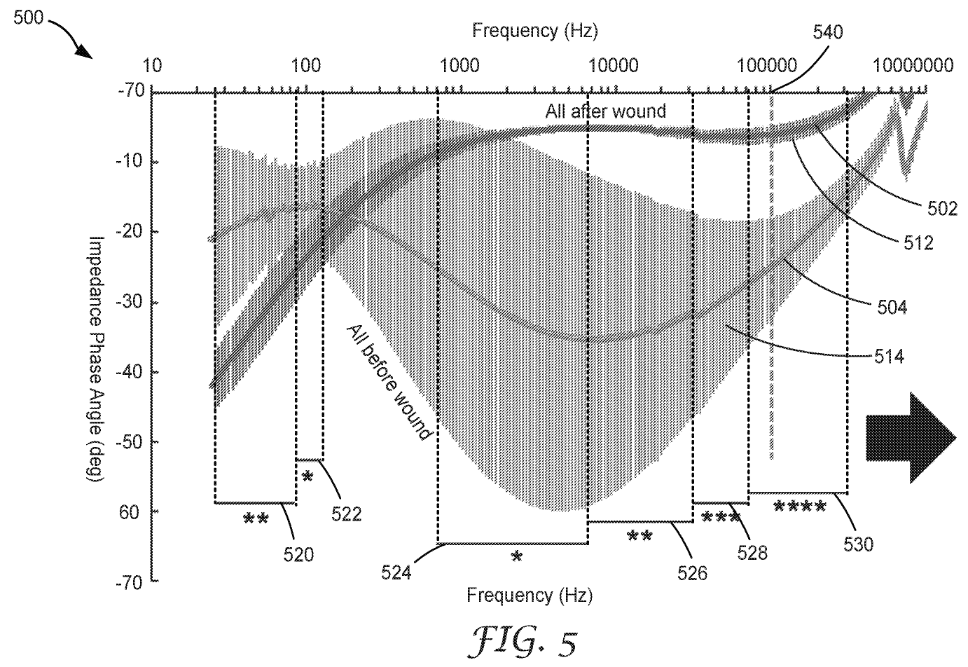

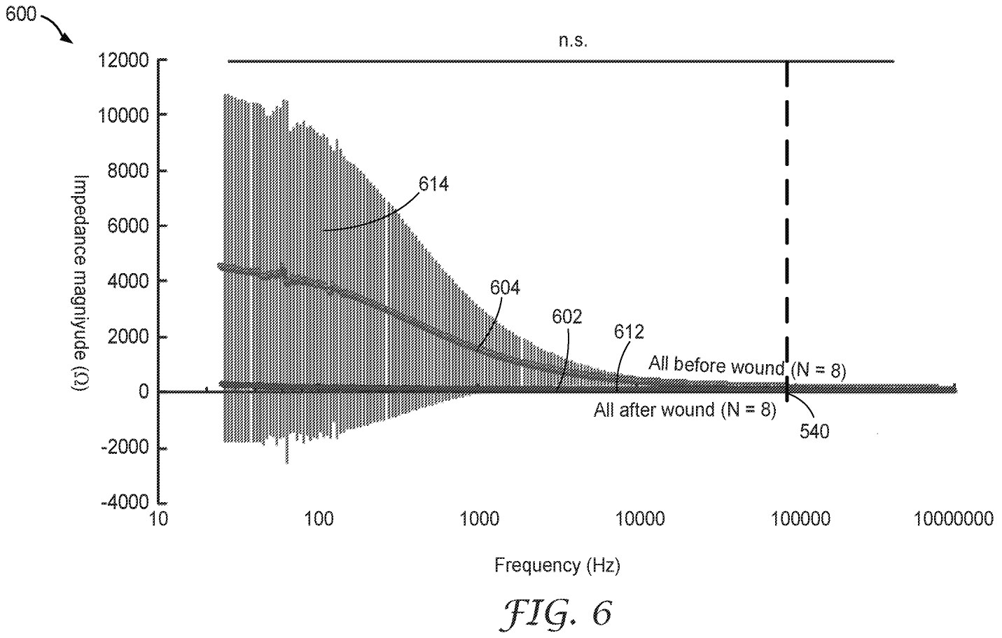

FIGS. 5-6 illustrate impedance phase angles and magnitudes of unwounded and wounded tissue as a function of frequency and will be described in conjunction below.

FIG. 5 is a plot 500 of example impedance phase angles as a function of signal frequency for first and second tissue sites, in accordance with the techniques described in this disclosure. The example shown includes first tissue impedance phase angle curve 502, corresponding to the mean of wounded tissue impedance phase angle curves collected over eight wounded tissue sites and second impedance phase angle curve 504 corresponding to the mean of baseline impedance phase angle curves collected over eight unwounded tissue sites.

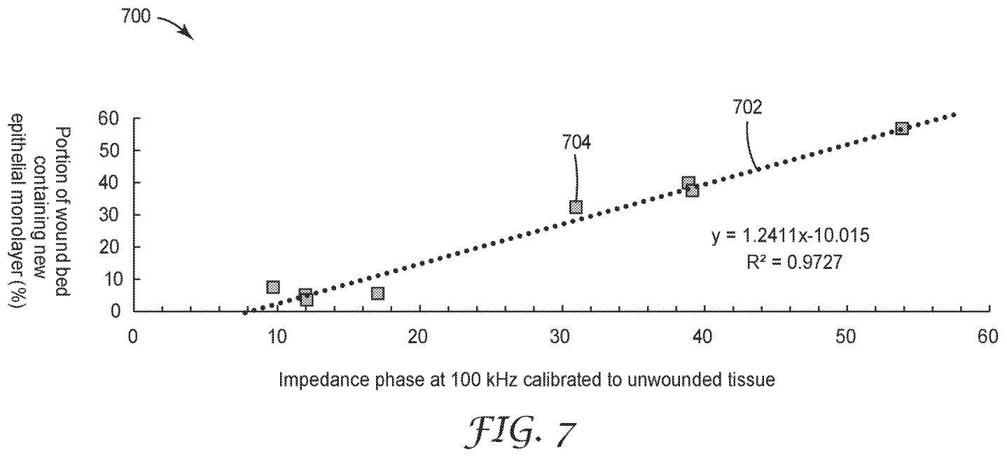

In examples, FIG. 5 illustrates that impedance phase angle measured at specific frequencies may be used to statistically distinguish between unwounded and wounded tissues across multiple tissue sites and across multiple animals. In the example shown, eight two-inch by two-inch tissue regions were designated on two different animals, e.g., four tissue areas on each individual animal, on animal backs flanking the animals’ medial planes. Impedance measurements of these eight unwounded tissue regions were collected by placing two sterile saline-laden gauze on the superior and inferior regions of each tissue region, and sterilized stainless-steel spatulas were placed on these gauzes to form electrical contact with an impedance analyzer. These represented unwounded baseline impedance measurements. Two-inch by two-inch partial thickness wounds were then generated in these regions using a dermatome. Five minutes were minutes were provided for the wounds to reach hemostasis. Impedance measurements of the wound were then collected by placing two sterile saline-laden gauze on the superior and inferior regions of the wound bed, and sterilized stainless-steel spatulas were placed on these gauzes to form electrical contact with an impedance analyzer. These represented impedance measurements for freshly wounded tissues. Impedance measurements were also at various time points after wounding, e.g., eighteen hours, three days, and seven days after wounding. These represented impedance measurements for wounds under various degrees of healing. After seven days, wound beds were excised, fixed, and histological sections were analyzed to quantify the percent new epithelium, epithelial cross-sectional area, epithelial depth, and corneal site de-nucleation of each section, with results illustrated and discussed below with respect to FIGS. 7, 8, 9B, 9C, 10B, 10C, 11B, and 11C.

In some examples, measurement of impedance phase angle of wounded tissue within particular frequency ranges may be distinguishable from measurement of impedance phase angle of unwounded tissue, and indistinguishable within other particular frequency ranges. For example, plot 500 includes baseline uncertainty area 514, which corresponds to an example standard deviation in measurement of impedance phase angle curve 504. Plot 500 also includes wounded tissue uncertainty area 512, which corresponds to an example standard deviation in measurement of wounded tissue impedance phase angle 502. In some examples, uncertainty in impedance phase angle measurements may be caused by differences in electrical characteristics of differing tissue locations within the same patient, or any animal, and differences in electrical characteristics of tissue of differing patients.

In the example shown, a wound may damage the inherent electrical capacitance of the tissue. For example, the impedance of the wounded, damaged tissue may be equivalent that of an electrical component without reactance, e.g., a resistor. As such, the impedance phase angle may tend towards zero, and the uncertainty of impedance phase angle between differing tissue locations and patients is significantly reduced. For example, uncertainty area 512 illustrated in FIG. 5 is significantly less than uncertainty area 514. In some examples, the change in impedance phase angle of wounded tissue versus unwounded tissue, along with a significant reduction in uncertainty of measurement of impedance phase angle of wounded tissue versus unwounded tissue, results in frequency ranges in which impedance phase angle may distinguish between wounded and unwounded tissue across a wide variety of tissue locations and patients.

In the example shown, frequency ranges 520-530 are frequency ranges in which a measurement of wounded tissue impedance phase angle 502 is statistically distinguishable from baseline impedance phase angle 504. For example, a calculated probability, e.g., a p-value, in frequency ranges 522 and 524 is less than 0.05 (p < 0.05), in frequency ranges 520 and 526 p < 0.01, in frequency range 528 p < 0.001, and in frequency range 530 p < 0.0001.

As described above, a wound may reduce the inherent electrical capacitance of tissue resulting in the impedance phase angle tending towards zero over a range of frequencies associated with capacitive charging of the epithelial layer. In some examples, as a wound heals, impedance phase angle curve 502 shifts towards impedance phase angle curve 504 as the tightly joined epithelial layers are gradually restored, thereby restoring the tissues inherent capacitance. In some examples, impedance phase angle measured using electrical signals within one or more frequency ranges, e.g., frequency ranges 520-530, may be used to determine information indicative of epithelial tissue characteristics. In other examples, impedance phase angle measured using electrical signals of a single frequency, e.g., frequency 540, may be used to determine information indicative of epithelial tissue characteristics. In still other examples, features of an impedance phase angle curve over a wide range of frequencies, e.g., impedance phase angle curve 502, may be determined and may be used to determine information indicative of epithelial tissue characteristics. For example, the values, curvature, inflexion points, rates of change, peak and valley locations, magnitudes, and widths, and the like, may be used to distinguish between wounded and unwounded tissue, as well as indicated an amount of healing.

FIG. 6 is a plot 600 of example impedance magnitudes as a function of signal frequency for first and second tissue sites, in accordance with the techniques described in this disclosure. The example shown includes first tissue impedance phase magnitude curve 602 corresponding to the mean of wounded tissue impedance magnitude curves collected over eight wounded tissue sites and second impedance phase magnitude curve 604 corresponding to the mean of baseline impedance magnitude curves collected over eight unwounded tissue sites.

In the example shown, and in contrast to impedance phase angle curves 502 and 504, measurement of impedance magnitude of wounded tissue may be indistinguishable from measurement of impedance magnitude over a broad range of frequencies. For example, plot 600 includes baseline uncertainty area 614, which corresponds to an example standard deviation in measurement of impedance phase magnitude curve 604. Plot 600 also includes wounded tissue uncertainty area 612, which corresponds to an example standard deviation in measurement of wounded tissue impedance magnitude 602. In some examples, uncertainty in impedance magnitude measurements may be caused by differences in electrical characteristics of differing tissue locations within the same patient, or any animal, and differences in electrical characteristics of tissue of differing patients. In the example shown, wounded tissue impedance phase angle 502 is not statistically distinguishable from baseline impedance phase angle 504 across the entire frequency range 10 Hz to 1 MHz.

FIG. 7 is a plot 700 of an example correlation between normalized impedance phase angle at a single frequency and a percentage of new epithelial monolayer, in accordance with the techniques described in this disclosure. In some examples, normalized impedance phase angle may indicate an amount of early stage healing, e.g., an amount of new epithelial monolayer growth of a wound site.

In some examples, impedance phase angle of wounded tissue may be normalized via division by the baseline impedance phase angle. For example, the value of impedance phase angle 502 at 100 kHz may be normalized, e.g., divided by the value of baseline impedance phase angle 504 at 100 kHz. In the example shown, normalized impedance phase angle may increase in proportion with new epithelial monolayer growth, and a value of normalized impedance phase angle may indicate an amount of new epithelial monolayer growth.

In the example shown, plot 700 includes a plurality of epithelial growth points 704 corresponding to a plurality of wound sites at which both impedance phase angle and a percentage of epithelial monolayer growth via histology are measured. Additionally, impedance phase angles corresponding to unwounded tissue were measured for each wound site and used to normalize/calibrate each wound site impedance phase angle measurement. In some examples, normalized impedance phase angle correlates strongly with new epithelial monolayer growth, as illustrated in the example shown via correlation curve 702.

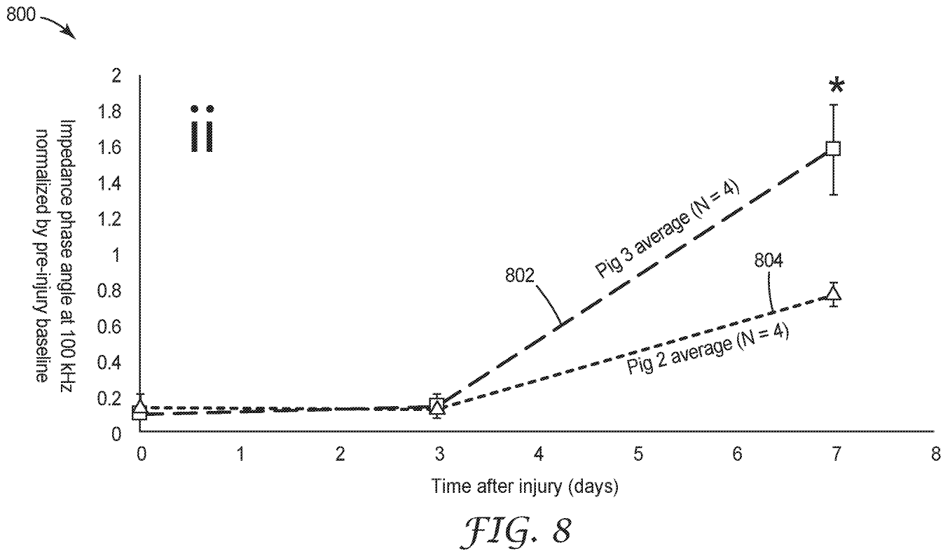

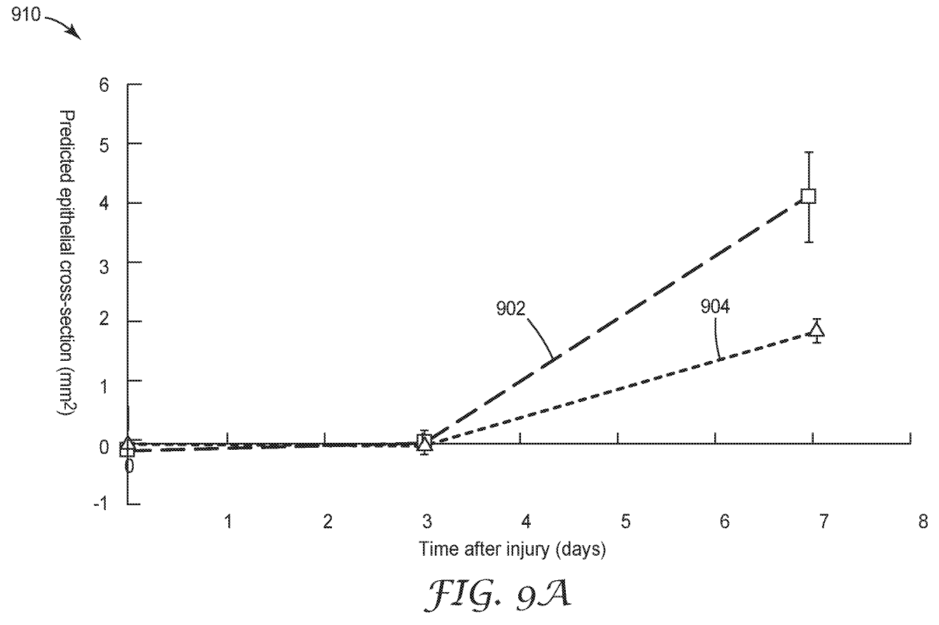

FIG. 8 is a plot 800 of example normalized impedance phase angles of wounded tissues versus time as eight wounds heal on two animals, in accordance with the techniques described in this disclosure Plot 800 includes mean normalized impedance phase angle curves 802 and 804 versus time, e.g., the number of days after wounding, ± standard deviation (error bars) for normalized phase angles measured at 100 kHz. Curve 802 corresponds to four wounds on an animal, and curve 804 corresponds to four wounds on a second animal that experienced complications that hindered healing. For example, the box plots of each of curves 802 and 804 correspond to four data points each. Both animals were elderly and did not exhibit strong healing until after day 3. In some examples, normalized impedance phase angle may indicate a delay in healing at an early stage, as indicated here by low normalized impedance readings on Day 3 for both curves 802 and 804.

In some examples, wound sites of interest corresponding to curve 802 may exhibit more epithelium cross-sectional area, average depth, and maturation, e.g., a more extensively developed stratum corneum evidenced via a greater percentage of the wound containing top layers of dead and de-nucleated cells, than wound sites of interest corresponding to curve 804 at day seven in plot 800. In some examples, normalized impedance phase angle may be used to track wound healing versus time and may be a metric usable to asses and/or compare relative rates and quality of healing.

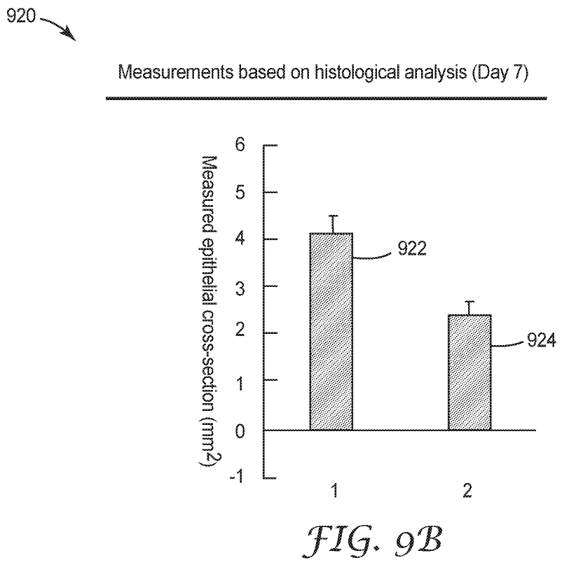

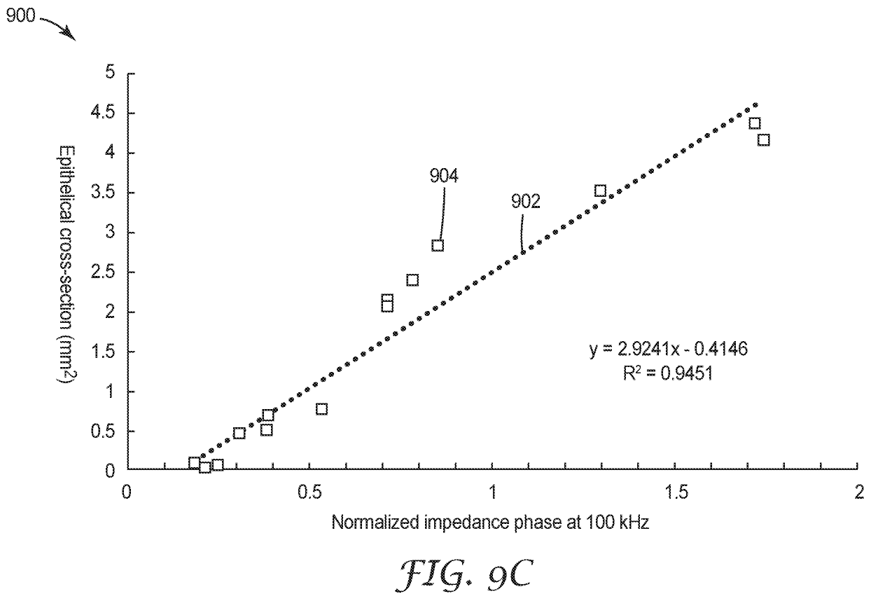

FIG. 9A is a plot 910 of example predicted epithelial cross-sectional areas as a function of time based on the normalized impedance phase angles versus time of FIG. 8, in accordance with the techniques described in this disclosure. Plot 910 includes predicted curves 912 and 914 based on normalized impedance phase angle curves 802 and 804, respectively. FIG. 9B is a plot 920 of example measured epithelial cross-sectional areas at day seven of FIG. 8 based on histology, in accordance with the techniques described in this disclosure. The examples of FIGS. 9A and 9B illustrate a correlation between predicted epithelial cross-sectional area based on measurement of normalized impedance phase angle and epithelial cross-sectional area of the same wounds based on an independent test, e.g., histology. For example, the Day 7 data points of predicted curves 912 and 914 of FIG. 9A substantially match the measured values of the bar graphs 922 and 924 of FIG. 9B, respectively.

FIG. 9C is a plot 900 of an example correlation between normalized impedance phase angle at a single frequency and epithelial cross-sectional area, in accordance with the techniques described in this disclosure. Plot 900 includes correlation curve 902 at 100 kHz. In some examples, normalized impedance phase angle may indicate an amount of healing at advanced stages, e.g., an amount of epithelial cross-sectional area growth of a wound site.

In the example shown, normalized impedance phase angle may increase in proportion with epithelial cross-sectional area growth, and a value of normalized impedance phase angle may indicate an amount of epithelial cross-sectional area growth.

In the example shown, plot 900 includes a plurality of epithelial cross-sectional area growth points 904 corresponding to a plurality of times at which both impedance phase angle and a percentage of epithelial cross-sectional area growth are measured. In some examples, impedance phase angle correlates strongly with epithelial cross-sectional area growth, as illustrated in the example shown via correlation curve 902.

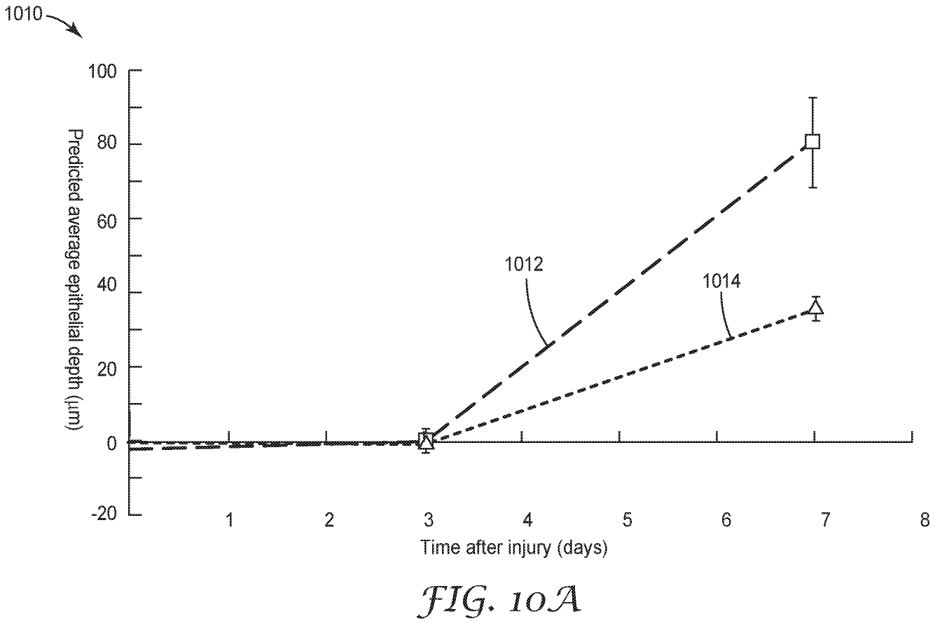

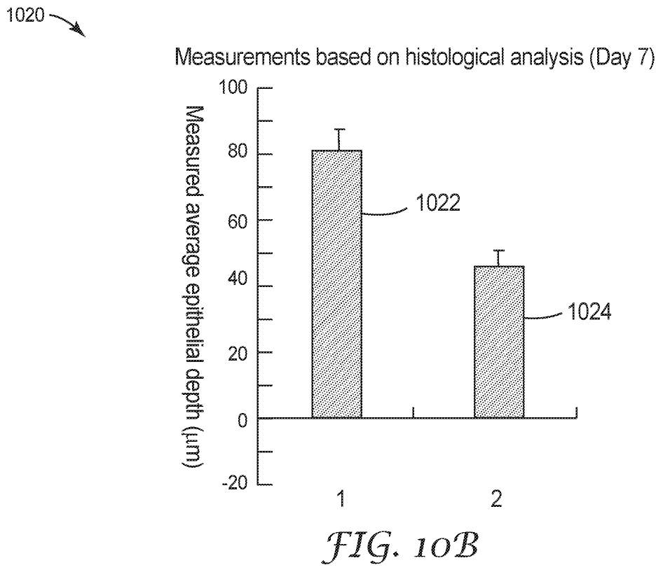

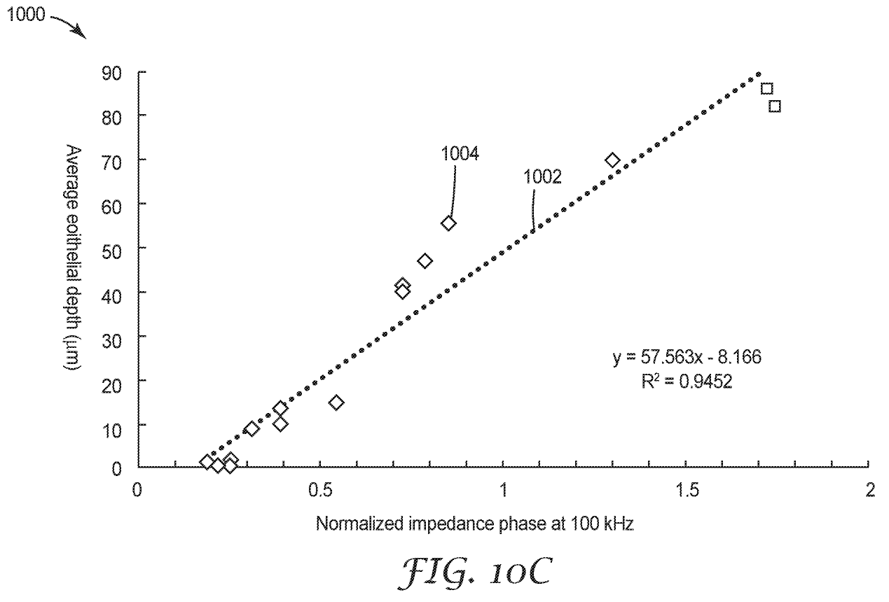

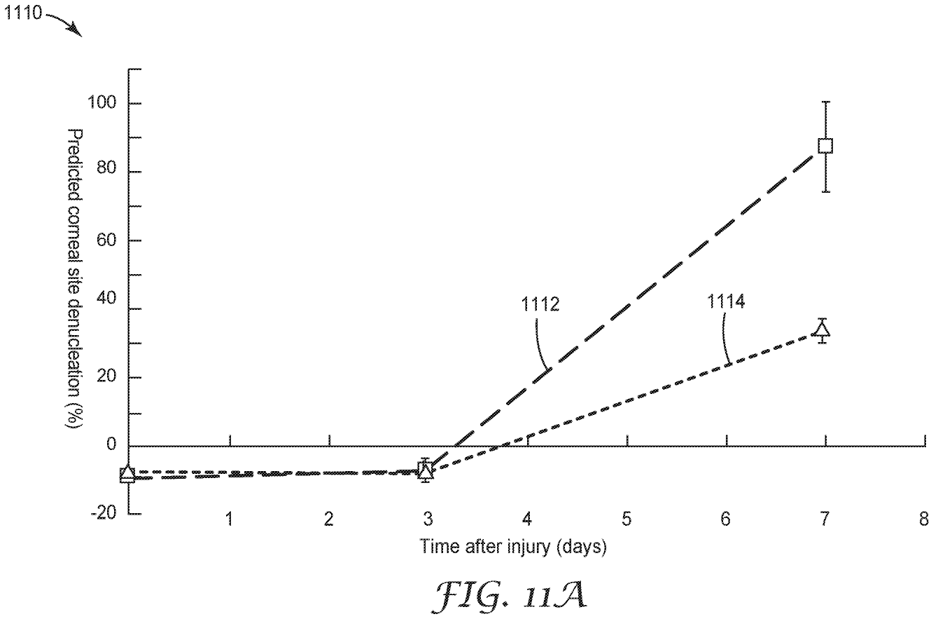

FIG. 10A is a plot 1010 of example predicted average epithelial depths as a function of time based on the normalized impedance phase angles versus time of FIG. 8, in accordance with the techniques described in this disclosure. Plot 1010 includes predicted curves 1012 and 1014 based on normalized impedance phase angle curves 802 and 804, respectively. FIG. 10B is a plot 1020 of example measured average epithelial depths at day seven of FIG. 8 based on histology, in accordance with the techniques described in this disclosure. The examples of FIGS. 10A and 10B illustrate a correlation between predicted average epithelial depth based on measurement of normalized impedance phase angle and average epithelial depth of the same wounds based on an independent test, e.g., histology. For example, the Day 7 data points of predicted curves 1012 and 1014 of FIG. 10A substantially match the measured values of the bar graphs 1022 and 1024 of FIG. 10B, respectively.

FIG. 10C is a plot 1000 of an example correlation between impedance phase angle at a single frequency and average epithelial depth, in accordance with the techniques described in this disclosure. Plot 1000 includes correlation curve 1002 at 100 kHz. In some examples, normalized impedance phase angle may indicate an amount of healing at advanced stages, e.g., an amount of average epithelial depth of a wound site.

In the example shown, normalized impedance phase angle may increase in proportion with average epithelial depth, and a value of normalized impedance phase angle may indicate an amount of average epithelial depth.

In the example shown, plot 1000 includes a plurality of average epithelial depth points 1004 corresponding to a plurality of times at which both impedance phase angle and an average epithelial depth are measured. In some examples, impedance phase angle correlates strongly with average epithelial depth, as illustrated in the example shown via correlation curve 1002.

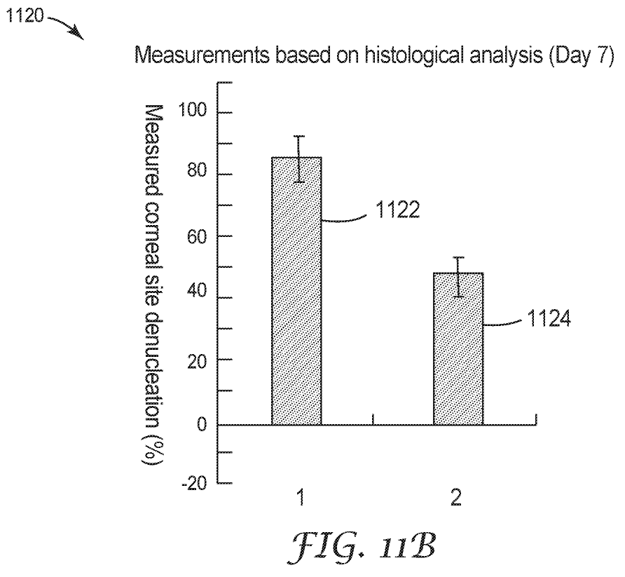

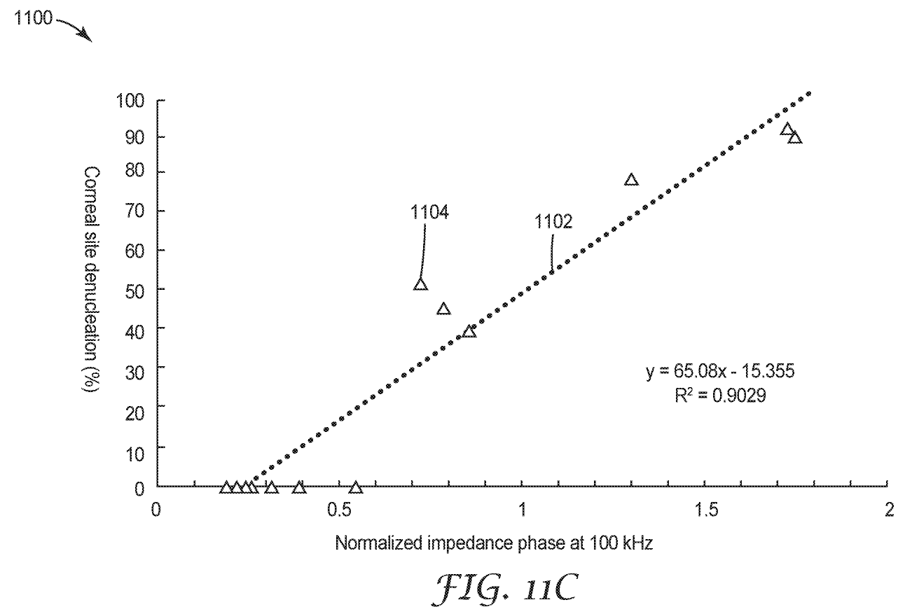

FIG. 11A is a plot 1110 of example predicted corneal site de-nucleation percentages as a function of time based on the normalized impedance phase angles versus time of FIG. 8, in accordance with the techniques described in this disclosure. Plot 1110 includes predicted curves 1112 and 1114 based on normalized impedance phase angle curves 802 and 804, respectively. FIG. 11B is a plot 1120 of example measured corneal site de-nucleation percentages at day seven of FIG. 8 based on histology, in accordance with the techniques described in this disclosure. The examples of FIGS. 11A and 11B illustrate a correlation between predicted percentage of corneal site de-nucleation based on measurement of normalized impedance phase angle and percentage of corneal site de-nucleation of the same wounds based on an independent test, e.g., histology. For example, the Day 7 data points of predicted curves 1112 and 1114 of FIG. 11A substantially match the measured values of the bar graphs 1122 and 1124 of FIG. 11B, respectively.

FIG. 11C is a plot 1100 of an example correlation between impedance phase angle at a single frequency and a percentage of corneal site de-nucleation, in accordance with the techniques described in this disclosure. Plot 1100 includes correlation curve 1102 at 100 kHz. In some examples, normalized impedance phase angle may indicate an amount of healing at advanced stages, e.g., an amount of corneal site de-nucleation of a wound site.

In the example shown, normalized impedance phase angle may increase in proportion with a percentage of corneal site de-nucleation, and a value of normalized impedance phase angle may indicate an amount of corneal site de-nucleation.

In the example shown, plot 1100 includes a plurality of percentage of corneal site de-nucleation points 1104 corresponding to a plurality of times at which both impedance phase angle and a percentage of corneal site de-nucleation are measured. In some examples, impedance phase angle correlates strongly with the percentage of corneal site de-nucleation, as illustrated in the example shown via correlation curve 1102.

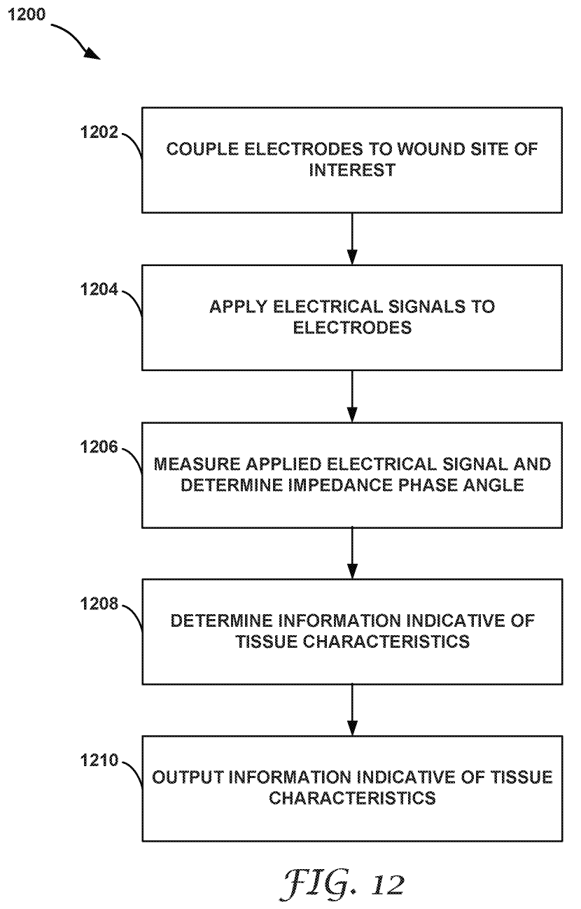

FIG. 12 is a flowchart of an example method of monitoring wound healing, in accordance with one or more techniques of this disclosure. The example method is described with reference to FIGS. 1-4. The example method may be performed, for example, by a computing device, such as computing device 106, executing the steps of the method.

A user, a patient, and/or a clinician may couple electrodes 130 and 132 to tissue site 150 (1202). In some examples, a user, a patient, and/or a clinician may couple electrodes 134 and 136 to tissue adjacent to tissue site 150, e.g., second tissue site 152. In some examples, electrodes 130 and 132 may be included in a dressing and attached to would site of interest 150 via the dressing. In some examples, a user may apply a material to electrodes 130-136 and/or tissue site 150 or tissue adjacent to tissue site 150 to improve electrical coupling between electrodes 130, 132, 134, and 136 and the tissue. In some examples, a user may couple a plurality of electrodes 330 to a tissue site 350 allowing for a plurality of applied electrical signals corresponding to a plurality of locations within tissue site 350 and measurement and determination of a plurality of impedance phase angles at the plurality of locations within tissue site 350.

Signal generator 402 may apply an electrical signal to tissue site 150 via electrodes 130 and 132 (1204). In some examples, signal generator 402 may apply an electrical signal, e.g., a baseline electrical signal, to tissue adjacent to tissue site 150 via electrodes 134 and 136. In some examples, signal generator 402 may apply the baseline electrical signal at substantially the same time as the electrical signal applied to tissue site 150, and in other examples signal generator 402 may apply the baseline electrical signal at a different time than the electrical signal applied to tissue site 150. In some examples, memory 124 and/or 224 and may store the baseline electrical signal, e.g., via memory 224 and/or 124. In some examples, signal generator 402 may apply a sinusoidal waveform electrical signal having a single frequency, e.g., substantially at or near 100 kHz, via electrodes 130-136. In other examples, signal generator 402 may apply a waveform that may differ from a sinusoidal waveform, e.g., a square wave, sawtooth wave, triangle wave, and the like, and a waveform that may include a plurality of frequencies.

In some examples, a plurality of electrical signals may be applied via electrodes 130-136, 330, and/or 334-336 at a plurality of predetermined times, e.g., allowing for measurement and/or determination of a plurality of impedance phase angles at the plurality of predetermined times so as to allow for tracking and/or monitoring of healing of a wound site.

In some examples, an electrical signal applied to tissue site 150 via electrodes 130 and 132 may be measured (1206), for example, via signal monitor 404. In some examples, a voltage signal is applied via electrodes 130-136, and signal monitor 404 measures a resulting current signal, e.g., current signals 422 and 424. An impedance phase angle may be determined based on the applied electrical signal (1206), e.g., via measurement of the resulting signal. For example, an impedance phase angle may be determined based on a phase delay between the applied voltage signal and the measured current signals.

Information indicative of epithelial tissue characteristics may be determined based on the impedance phase angle or angles (1208). For example, information indicative of an amount of healing, an amount of new epithelial monolayer regenerated in tissue, an amount of size and thickness of regenerated epithelium, an amount of maturation of stratus corneum, an amount or percentage of corneal site de-nucleation, and the like may be determined based on normalized impedance phase angle. In some examples, impedance phase angle of wounded tissue may be normalized via division by impedance of unwounded tissue, e.g., as a ratio of the impedance phase angle of wounded tissue to the impedance phase angle of unwounded tissue.

Information indicative of epithelial tissue characteristics may be output (1210), e.g., to computing device 106. For example, electrodes 130-136, or electrodes 330, 334, and 336, may be communicatively coupled to computing device 106, or to processing circuitry 116.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples, a non-transitory storage medium may store data that can, over time, change (e.g., in RAM or cache).

The following examples may illustrate one or more aspects of the disclosure:

Example 1. A system for tissue impedance measurement, the system comprising: electrical contacts configured to be coupled to a first tissue; a first device configured to apply a first electrical signal to the first tissue via the electrical contacts; and a second device configured to: determine a first impedance phase angle of epithelial tissue of the first tissue site based on the first applied electrical signal; determine a baseline impedance phase angle of epithelial tissue corresponding to a second tissue; determine information indicative of epithelial tissue characteristics based on a ratio of the first impedance phase angle and the baseline impedance phase angle; and output information indicative of the epithelial tissue characteristics.

Example 2. The system of claim 1, wherein to determine the first impedance phase angle, the second device is configured to determine a phase angle of a voltage of the first applied electrical signal and a phase angle of a current of the first applied electrical signal, and determine the impedance phase angle based on a difference in the phase angle of the voltage of the first applied electrical signal and the phase angle of the current of the first applied electrical signal.

Example 3. The system of any of claims 1 and 2, wherein the electrical contacts comprise a first set of electrical contacts, wherein the first device is further configured to apply a second electrical signal through the second tissue via a second set of electrical contacts, and wherein the second device is configured to determine the baseline impedance phase angle based on the second applied electrical signal, and

Example 4. The system of any of claims 1–3, wherein to apply the first electrical signal, the first device is configured to apply a sinusoidal wave at a predetermined single frequency, and wherein to determine the first impedance phase angle, the second device is configured to determine the impedance phase angle at the predetermined frequency.

Example 5. The system of claim 4, wherein the single frequency is in range of approximately 10 kHz to 300 kHz.

Example 6. The system of claim 4, wherein the single frequency is approximately 100 kHz.

Example 7. The system of any of claims 1–6, wherein the information indicative of epithelial tissue characteristics comprises information indicative of an amount of healing.

Example 8. The system of claim 7, wherein the information indicative of the amount of healing comprises one or more of an amount of new epithelial monolayer regenerated in the tissue, amount of size or thickness of regenerated epithelium, or relative maturation of stratus corneum.

Example 9. The system of any of claims 1–8, wherein information indicative of epithelial tissue characteristics based on a ratio of the first impedance phase angle and the baseline impedance phase angle is determined without using a magnitude of the first applied electrical signal.

Example 10. The system of any of claims 1–9, wherein the first device is configured to apply a plurality of first electrical signals through the wounded tissue via the electrical contacts, each signal being applied at each of a plurality of predetermined times over a period of time, wherein the second device is configured to: determine a plurality of impedance phase angles of the wounded tissue based on the corresponding applied first electrical signals at each of the plurality of predetermined times; and determine information indicative of epithelial tissue characteristics based on a plurality of ratios of the plurality of first impedance phase angles and one or more baseline impedance phase angles.

Example 11. The system of any of claims 1–10, wherein the first device is configured to apply a plurality of first electrical signals through the wounded tissue via the electrical contacts, each signal being applied at each of a plurality of predetermined locations within a wounded tissue area, wherein the second device is configured to: determine a plurality of impedance phase angles of the wounded tissue corresponding to the plurality of predetermined locations and based on the corresponding plurality of applied first electrical signals; and determine information indicative of epithelial tissue characteristics based on a plurality of ratios of the plurality of first impedance phase angles and one or more baseline impedance phase angles.

Example 12. The system of any of claims 1–11, wherein the first device and the second device are a single device.

Example 13. A method, comprising: applying a first electrical signal through a first tissue via electrical contacts; determining a first impedance phase angle of the first tissue based on the first applied electrical signal; determining a baseline impedance phase angle corresponding to a second tissue; determining information indicative of epithelial tissue characteristics based on a ratio of the first impedance phase angle and the baseline impedance phase angle; and outputting information indicative of the tissue characteristics.

Example 14. The method of claim 13, wherein applying the first electrical signal comprises applying a sinusoidal wave at a predetermined single frequency, and wherein determining the first impedance phase angle comprises determining the impedance phase angle at the predetermined frequency.

Example 15. The method of claim 14, wherein the single frequency is in range of approximately 10 kHz to 300 kHz.

Example 16. The system of claim 13, the information indicative of epithelial tissue characteristics comprises information indicative of an amount of healing.

Example 17. The method of claim 16, wherein the information indicative of the amount of healing comprises one or more of an amount of new epithelial monolayer regenerated in the tissue, amount of size or thickness of regenerated epithelium, or relative maturation of stratus corneum.

Example 18. The method of claim 13, further comprising: applying a plurality of first electrical signals through the first tissue via the electrical contacts, each first signal being applied at each of a plurality of predetermined times; determining a plurality of first impedance phase angles of the first tissue corresponding to the plurality of predetermined times and based on the plurality of applied first electrical signals; and determine information indicative of epithelial tissue characteristics based on a plurality of ratios of the plurality of first impedance phase angles and the baseline impedance phase angle.

Example 19. The method of claim 13, further comprising: applying a plurality of first electrical signals through the first tissue via the electrical contacts, each first signal being applied at each of a plurality of predetermined locations within a first tissue site; determining a plurality of first impedance phase angles of the first tissue corresponding to the plurality of predetermined locations and based on the plurality of applied first electrical signals; and determine information indicative of epithelial tissue characteristics based on a plurality of ratios of the plurality of first impedance phase angles and the baseline impedance phase angle.

Example 20. A dressing, comprising: electrical contacts configured to be coupled to wounded tissue and to unwounded tissue proximate to wounded tissue; a first device configured to apply a first electrical signal through the wounded tissue via the electrical contacts, the first device further configured to apply a second electrical signal through the unwounded tissue; and a second device configured to: determine a first impedance phase angle of the tissue based on the first applied electrical signal; determine a second impedance phase angle off the tissue based on the second applied electrical signal; determine information indicative of epithelial tissue characteristics based on a ratio of the first impedance phase angle and the second impedance phase angle; and output information indicative of the tissue characteristics.

Various examples have been described. These and other examples are within the scope of the following claims.

1. A system for tissue impedance measurement, the system comprising:
electrical contacts configured to be coupled to a first tissue;
a first device configured to apply a first electrical signal to the first tissue via the electrical contacts; and
a second device configured to:
determine a first impedance phase angle of epithelial tissue of the first tissue site based on the first applied electrical signal;
determine a baseline impedance phase angle of epithelial tissue corresponding to a second tissue;
determine information indicative of epithelial tissue characteristics based on a ratio of the first impedance phase angle and the baseline impedance phase angle; and
output information indicative of the epithelial tissue characteristics.

2. The system of claim 1, wherein to determine the first impedance phase angle, the second device is configured to determine a phase angle of a voltage of the first applied electrical signal and a phase angle of a current of the first applied electrical signal, and determine the impedance phase angle based on a difference in the phase angle of the voltage of the first applied electrical signal and the phase angle of the current of the first applied electrical signal.

3. The system of claim 1,
wherein the electrical contacts comprise a first set of electrical contacts, wherein the first device is further configured to apply a second electrical signal through the second tissue via a second set of electrical contacts, and
wherein the second device is configured to determine the baseline impedance phase angle based on the second applied electrical signal.

4. The system of claim 1, wherein to apply the first electrical signal, the first device is configured to apply a sinusoidal wave at a predetermined single frequency, and wherein to determine the first impedance phase angle, the second device is configured to determine the impedance phase angle at the predetermined frequency.

5. The system of claim 4, wherein the single frequency is in range of approximately 10 kHz to 300 kHz.

6. The system of claim 4, wherein the single frequency is approximately 100 kHz.

7. The system of claim 1, wherein the information indicative of epithelial tissue characteristics comprises information indicative of an amount of healing.

8. The system of claim 7, wherein the information indicative of the amount of healing comprises one or more of an amount of new epithelial monolayer regenerated in the tissue, amount of size or thickness of regenerated epithelium, or relative maturation of stratus corneum.

9. The system of claim 1, wherein information indicative of epithelial tissue characteristics based on a ratio of the first impedance phase angle and the baseline impedance phase angle is determined without using a magnitude of the first applied electrical signal.

10. The system of claim 1, wherein the first device is configured to apply a plurality of first electrical signals through the wounded tissue via the electrical contacts, each signal being applied at each of a plurality of predetermined times over a period of time, wherein the second device is configured to:
determine a plurality of impedance phase angles of the wounded tissue based on the corresponding applied first electrical signals at each of the plurality of predetermined times; and
determine information indicative of epithelial tissue characteristics based on a plurality of ratios of the plurality of first impedance phase angles and one or more baseline impedance phase angles.

11. The system of claim 1, wherein the first device is configured to apply a plurality of first electrical signals through the wounded tissue via the electrical contacts, each signal being applied at each of a plurality of predetermined locations within a wounded tissue area, wherein the second device is configured to:
determine a plurality of impedance phase angles of the wounded tissue corresponding to the plurality of predetermined locations and based on the corresponding plurality of applied first electrical signals; and
determine information indicative of epithelial tissue characteristics based on a plurality of ratios of the plurality of first impedance phase angles and one or more baseline impedance phase angles.

12. The system of claim 1, wherein the first device and the second device are a single device.

13. A method, comprising:
applying a first electrical signal through a first tissue via electrical contacts;
determining a first impedance phase angle of the first tissue based on the first applied electrical signal;
determining a baseline impedance phase angle corresponding to a second tissue;
determining information indicative of epithelial tissue characteristics based on a ratio of the first impedance phase angle and the baseline impedance phase angle; and
outputting information indicative of the tissue characteristics.

14. The method of claim 13, wherein applying the first electrical signal comprises applying a sinusoidal wave at a predetermined single frequency, and wherein determining the first impedance phase angle comprises determining the impedance phase angle at the predetermined frequency.

15. The method of claim 14, wherein the single frequency is in range of approximately 10 kHz to 300 kHz.

16. The system of claim 13, the information indicative of epithelial tissue characteristics comprises information indicative of an amount of healing.

17. The method of claim 16, wherein the information indicative of the amount of healing comprises one or more of an amount of new epithelial monolayer regenerated in the tissue, amount of size or thickness of regenerated epithelium, or relative maturation of stratus corneum.

18. The method of claim 13, further comprising:
applying a plurality of first electrical signals through the first tissue via the electrical contacts, each first signal being applied at each of a plurality of predetermined times;
determining a plurality of first impedance phase angles of the first tissue corresponding to the plurality of predetermined times and based on the plurality of applied first electrical signals; and
determine information indicative of epithelial tissue characteristics based on a plurality of ratios of the plurality of first impedance phase angles and the baseline impedance phase angle.

19. The method of claim 13, further comprising:
applying a plurality of first electrical signals through the first tissue via the electrical contacts, each first signal being applied at each of a plurality of predetermined locations within a first tissue site;
determining a plurality of first impedance phase angles of the first tissue corresponding to the plurality of predetermined locations and based on the plurality of applied first electrical signals; and
determine information indicative of epithelial tissue characteristics based on a plurality of ratios of the plurality of first impedance phase angles and the baseline impedance phase angle.

20. A dressing, comprising:
electrical contacts configured to be coupled to wounded tissue and to unwounded tissue proximate to wounded tissue;
a first device configured to apply a first electrical signal through the wounded tissue via the electrical contacts, the first device further configured to apply a second electrical signal through the unwounded tissue; and
a second device configured to:
determine a first impedance phase angle of the tissue based on the first applied electrical signal;
determine a second impedance phase angle off the tissue based on the second applied electrical signal;
determine information indicative of epithelial tissue characteristics based on a ratio of the first impedance phase angle and the second impedance phase angle; and
output information indicative of the tissue characteristics.
